(19) United States
(12) Patent Application Publication
(10) Pub. No.: US 2003/0055382 A1
(43) Pub. Date: Mar. 20, 2003

(54) Intravenous catheter support

(76) Inventor: Rodney D. Schaeffer, Pennsylvania Furnace, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 09/954,783

(22) Filed: Sep. 18, 2001

(51) Int. Cl. A61M 5/32

(52) U.S. Cl. 604/179

(57) ABSTRACT

A device for securing an intravenous (IV) catheter inserted into a body portion of a patient includes a planar base member for placement on the body portion. An opening is formed in the base member and first and second hook-shaped members extend into the first opening in opposing relationship. The hook-shaped members are adapted to receive and hold an IV line of the catheter against movement. A pair of straps are connected to the base member for wrapping around the body member to secure the base member and the catheter to the body

### BACKGROUND OF THE INVENTION

This invention relates generally to intravenous (IV) devices, and more particularly to a device that can be quickly positioned on and secured to a body portion to protect and support an IV catheter inserted into a vein of a patient.

When an IV catheter is inserted into a vein of a patient, it is necessary to stabilize the position of the catheter with respect to the vein to ensure its proper operation during intravenous delivery of medication, nutrients, blood, or measurement of one or more physical parameters of the patient. In order to secure inserted IV catheters, medical personnel generally affix one or more lengths of adhesive tape to hold the catheter in place and to prevent foreign contaminants from contacting the entry point on the skin of the patient. Unfortunately, such taping can be less than adequate, and offers little in protection against mishaps that may occur due to patient moving or when the patient's bed clothing is changed. Dislodgment of the catheter from the designated vein can thus easily occur, resulting in tearing of the vein and painful injury of surrounding tissue, tearing of the skin in the elderly, as well as disruption of the intravenous therapy in all too often critical situations. Consequently, medical personnel must first become aware of the dislodgment, locate another vein, and re-insert the catheter. The number of veins available for intravenous therapy is, however, limited by both the patient's health condition and the number of veins already damaged by previous catheter insertions. It is often difficult, therefore, to re-insert a catheter into a patient who is in poor health or who has undergone extended intravenous therapy.

Although many solutions have been proposed to overcome the problems associated with IV catheter support, some prior art devices, such as splints or arm boards, are utilized to maintain the catheter in position by restricting limb flexure. Such boards cover a significant portion of the limb and thus greatly limit the number and location of potential catheter insertion sites. Such limitations may be less than desirable for patients having few available veins or requiring extended intravenous therapy.

In situations such as mass disasters or military field conditions, or when faced with treating single patients with life threatening injuries under extreme time constraints, medical personnel have found that the piecemeal traditional methods of inserting and supporting IV catheters are less than desirable.

### SUMMARY OF THE INVENTION

According to the invention, a device for securing an intravenous (IV) catheter inserted into a body portion of a patient is disclosed. The device comprises a generally longitudinal, substantially planar base member that is adapted for placement on a body portion of a patient. A first opening is formed in the base member and first and second hook-shaped members extend generally longitudinally into the first opening from the base member. The hook-shaped members are adapted to receive and hold an IV line of the catheter against movement. A first strap is connected to the base member and is adapted for wrapping around the body member to thereby secure the base member and the catheter to the body member.

Further according to the invention, a device for securing an intravenous (IV) catheter inserted into a body portion of a patient comprises a generally longitudinal, substantially planar base member adapted for placement on a body portion of a patient. A first opening is formed in the base member and includes a front edge, a rear edge, and a pair of side edges extending between the front and rear edges. A first hook-shaped member extends generally longitudinally into the opening from the front edge and a second hook-shaped member extends generally longitudinally into the opening from the rear edge in opposing relationship to the first hook-shaped member. The hook-shaped members are adapted to receive and hold the IV line against movement. A second opening is formed in the base member for receiving the catheter and comprises a circular edge and a pair of converging edges that extend from a front edge of the base member to the circular edge. First and second straps are connected to the base member and are adapted for wrapping around the body member to thereby secure the base member and the catheter to the body member. An adhesive strip is positioned on the base member for covering the second opening and securing the catheter to the base member.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

Embodiments of the present invention will hereinafter be described in conjunction with the appended drawings, wherein:

FIG. 1 is a top plan view of an intravenous catheter support according to an embodiment of the present invention;

FIG. 2 is a perspective view of the intravenous catheter support of FIG. 1 in a condition prior to use;

FIG. 3 is a side elevational view of a portion of the intravenous catheter support of FIG. 1; and

FIG. 4 illustrates the intravenous catheter support of FIG. 1 and an intravenous catheter installed on a body part.

Embodiments of the present invention will now be described in greater detail with reference to the drawings, wherein like parts throughout the drawing figures are represented by like numerals.

### DETAILED DESCRIPTION OF THE INVENTION

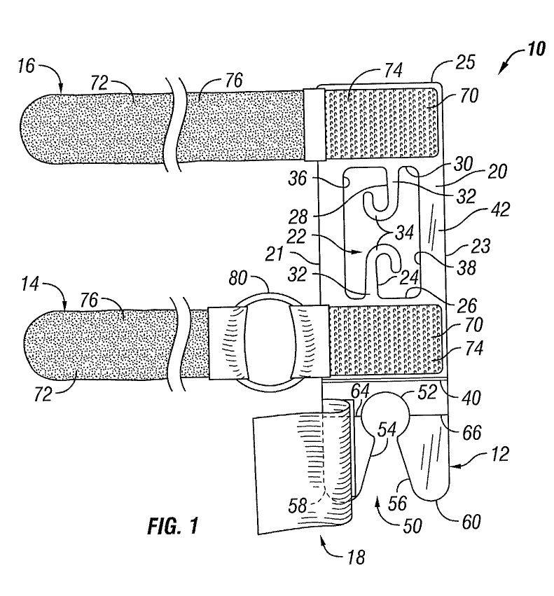

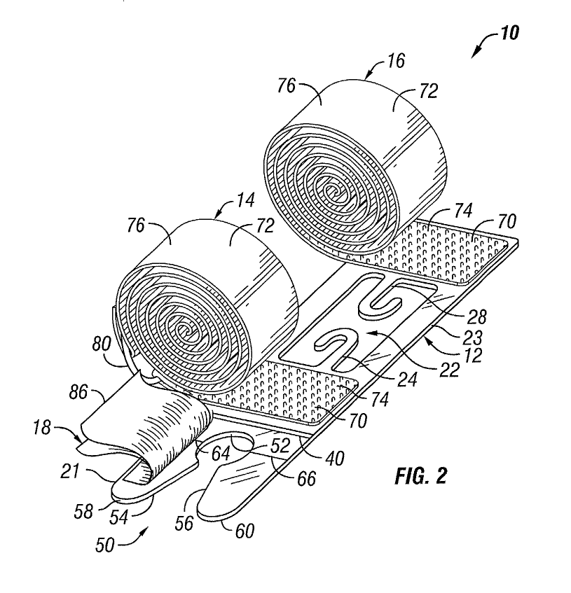

Referring now to the drawings, and to FIGS. 1 and 2 in particular, an intravenous (IV) catheter support 10 in accordance with an embodiment of the invention is illustrated. The IV catheter support 10 includes a base member 12 with a front mounting strap 14 and a rear mounting strap 16 connected to the base member 12 at spaced locations. An adhesive strip 18 is connected to the base member 12 forward of the front strap 14.

The base member 12 is preferably constructed of a planar sheet 20 of plastic or other material that is sufficiently flexible to conform to the contour of a patient's limb, such as the hand 84, wrist 88, and/or arm 90, as illustrated in FIG. 4, yet sufficiently stiff to support the IV catheter during use. The sheet 20 is preferably transparent or translucent in nature so that the underlying skin can be viewed both during and after securement of the catheter support 10 to the patient's limb to monitor the condition of the catheter insertion site, as well as the surrounding skin.

An elongate rear opening 22 is formed in the sheet 20 approximately midway between elongate side edges 21, 23 and proximal a rear edge 25 of the base member 12. A front hook 24 extends rearwardly from a front edge 26 of the opening 22 and a rear hook 28 extends forwardly from a rear edge 30 of the opening. Preferably, the hooks 24 and 28 are integrally formed with the sheet 20 and include a straight portion 32 extending from one of the edges 26, 30 and a curved portion 34 integrally formed with the straight portion 32. As shown in FIG. 1, the curved portion 34 of the hook 24 preferably extends to the right, while the curved portion 34 of the hook 28 preferably extends to the left. It will be understood, however, that the curved portion of the hook 24 can extend to the left, and that the curved portion of the hook 28 can extend to the right. The hooks 24 and 28 are preferably spaced a sufficient distance from the elongate edges 36, 38 of the opening 22 to permit insertion of an IV line or tubing 48 (FIG. 4) between the hooks and the elongate edges 36, 38. The hooks are preferably sufficiently flexible to permit insertion of the IV line 48 through the hooks, as shown in FIG. 4.

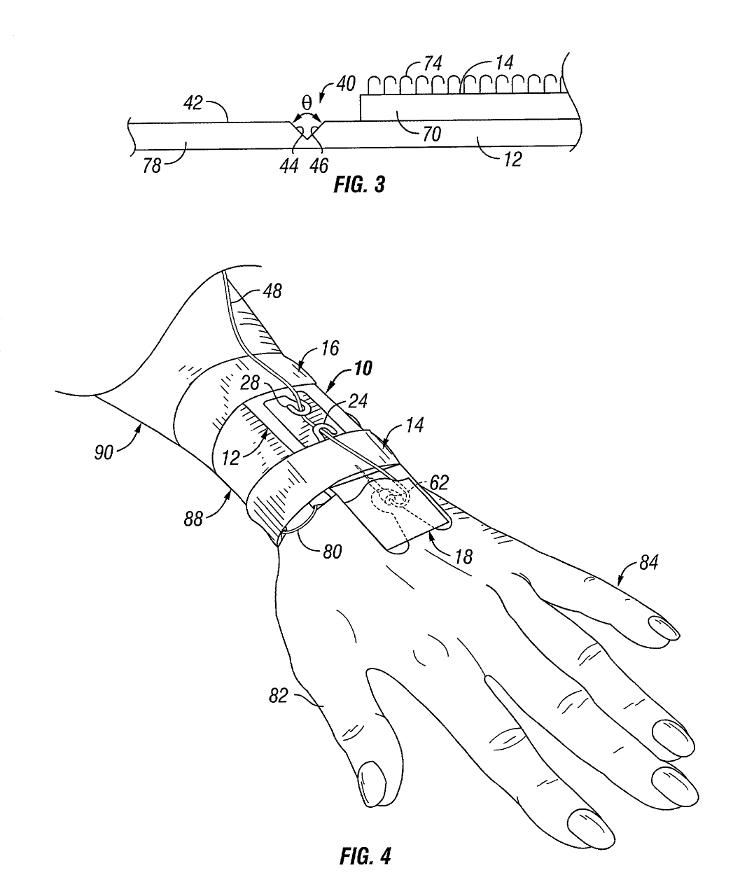

With additional reference to FIG. 3, a groove 40 is formed in an upper surface 42 of the base member 12. Preferably, the groove 40 extends between the elongate side edges 21 and 23 (FIG. 1) of the base member 12 and includes a first surface 44 and a second surface 46 that preferably form an angle θ of approximately 90°. The groove 40 enables the base member 12 to bend with arm movement and ensures that the area between the groove 40 and front edges 54, 56 (FIG. 1) of the base member remains in contact with the wrist or other body area.

Referring again to FIGS. 1 and 2, a front key-hole shaped opening 50 preferably includes a circular edge 52 located approximately midway between the elongate side edges 21 and 23 of the base member 12, and side edges 54, 56 that preferably converge radially toward a center point of the circular edge 52 from the curved front edges 58 and 60, respectively, of the base member 12. The curved front edges 58 and 60 together with the converging edges 54 and 56 facilitate positioning the catheter support 10 around a catheter 62 (FIG. 4) that has been installed in a body part. Alignment marks 64 and 66 are located on the base member 12 and preferably extend radially toward the center point of the circular edge 52 from the elongate side edges 21 and 23, respectively. The marks 64 and 66 facilitate proper placement of the IV catheter support with respect to the puncture site of the installed catheter.

The straps 14 and 16 are preferably mounted to the upper surface 42 of the base member 12, with the front strap 14 positioned between the groove 40 and the front edge 26 of the rear opening 22 and the rear strap 16 positioned between the rear edge 30 of the rear opening 22 and the rear edge 25 of the base member 12. The straps are preferably secured to the upper surface 42 through adhesive, although double-sided tape, stitching, ultrasonic welding, or any other known fastening technique can be used.

The straps 14 and 16 are preferably constructed of woven nylon or other sturdy material that normally exhibits little elongation when tensioned. The straps are sufficiently long to completely wrap around the body portion, and preferably sufficiently wide to prevent undue deformation of the skin on the body portion or other undue irritation.

Preferably, each strap 14, 16 has a fixed portion 70 which is secured directly to the base member 12 and a free portion 72 extending from the fixed portion 70. As shown in FIG. 2, the free portions 72 can be initially pre-rolled and detachably connected to their respective fixed portions 70. Preferably, hook material 74 is associated with the fixed portions 70 and loop material 76 is associated with the free portions 72 so that engagement of the hooks and loops detachably connects the free portions to the fixed portions in a well-known manner. It will be understood that the hook and loop material can be reversed on the fixed portions and free portions. It will be further understood that other means for fastening the free portions of the straps to the base member 12 can be used, such as adhesive, snaps, buckles, and so on.

In the pre-rolled condition, the straps 14, 16 do not interfere with a lower contact surface 78 (FIG. 3) of the base member 12 so that the IV catheter support 10 can be easily positioned on the body portion of a patient and aligned with the catheter 62 without undue attention to loose straps. The pre-rolled free portions 72 can then be quickly unrolled after being released, passed around the body portion, and secured to their respective fixed portions 70 to thereby secure the IV catheter support to the body portion.

Preferably, the front strap 14 includes a resilient ring 80 that connects the fixed portion 70 with the free portion 72. The ring 80 is positioned on the front strap 14 so that the strap 14 can be pivoted with respect to the base member 12 to fit the body part. As shown, the ring 80 is positioned rearwardly of the thumb 82 of a hand 84. However, the ring 80 may be positioned on the thumb, between the thumb and index finger, or at any other location depending on the position of the catheter support 10 with respect to the body part. With the ring 80 positioned between the thumb 82 and the index finger, the front strap 14 can secure the base member and the IV catheter 62 to the hand 84 while at the same time allowing the thumb 82 a degree of mobility without unduly disturbing the secured IV catheter 62. Alternatively, the front strap 14 can be constructed identical to the rear strap 16. The resilient ring 80 is preferably constructed of a silicone based material that provides tension to the strap 14 for purposes of securely mounting the base member 12 to the body part and allows flexibility in the event of body part increases in size due to inflammation or other medical type event.

The adhesive strip 18 is preferably longer than a width of the base member 12 so that when the IV catheter support 10 is installed, the adhesive strip will wrap over and adhere to the catheter 62 and terminate adjacent the elongate side edges 21 and 23 of the base member 12. A portion of the strip 18 may also adhere to the patient's body part in the location of the front opening 50. The adhesive strip 18 is also preferably constructed of a transparent or translucent material in order to view the catheter 62 and surrounding skin underneath. A backing layer 86 can be provided on a portion of the adhesive strip where it is not initially attached to the base member 12 as shown in FIG. 2, to protect the adhesive layer from contamination prior to installation. The adhesive strip 18 not only secures the catheter to the base member, but also covers and prevents infection over the puncture site.

In use, and by way of example, the IV catheter 62 is installed on a limb of the patient in a well-known manner. The IV support is then positioned on the patient's limb with the alignment marks 64, 66 preferably in alignment with the catheter insertion site. The straps 14 and 16 are then unrolled and the free portions 72 are wrapped around the patient's limb and secured to the base member 12 by engaging the loop material 76 with the hook material 74. The resistance to stretching of the strap material facilitates proper wrapping of the straps around the patient's limb without impeding blood flow through the limb. In order to secure the catheter 62 to the IV support 10, the backing layer 86 is removed from the adhesive strip 18 and the adhesive strip is positioned over the catheter 62 in the opening 50 and adhered to the upper surface 42 of the base member 12 on the opposite side of the opening 50. In order to secure the IV tubing 48 to the IV support 10, the hooks 24, 28 are slightly raised and the tubing 48 is positioned under the hooks. When released, the hooks will tend to spring back toward their initial position, due to the spring-like material property of the base member 12, to engage and firmly press against the tubing in a manner as shown in FIG. 4. The opposing orientation of the hooks 24, 28 secures the IV tubing against lateral movement in a quick and easy manner and thus makes it more difficult for the IV catheter 62 to be inadvertently dislodged.

It will be understood that the terms front, rear, side, forward, left, right, upper, lower, and their respective derivatives and equivalent terms as may be used throughout the specification, refer to relative, rather than absolute orientations and/or positions.

While the invention has been taught with specific reference to the above-described embodiments, those skilled in the art will recognize that changes can be made in form and detail without departing from the spirit and the scope of the invention. By way of example, although the opening 22 is shown as rectangular, the opening can be circular, square, or any other shape to accommodate the opposing nature of the hooks. In an alternative embodiment, the hooks may extend into the opening 22 from opposite elongate edges 36 and 38 of the opening. Thus, the described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A device for securing an intravenous (IV) catheter inserted into a body portion of a patient, the IV catheter being attached to an IV line, the device comprising:
a generally longitudinal, substantially planar base member adapted for placement on a body portion of a patient;
a first opening formed in the base member;
first and second hook-shaped members extending into the first opening from the base member, the hook-shaped members being adapted to receive and hold the IV line against movement; and
a first strap connected to the base member, the first strap being adapted for wrapping around the body member to thereby secure the base member and the catheter to the body member.

2. A device according to claim 1, wherein the first and second hook-shaped members extend toward each other.

3. A device according to claim 2, wherein the first opening includes a front edge, a rear edge, and a pair of side edges extending between the front and rear edges, and further wherein the first hook-shaped member extends from the front edge toward the rear edge and the second hook-shaped member extends from the rear edge toward the front edge, such that the hook-shaped members are in opposing relation.

4. A device according to claim 3, wherein the first hook-shaped member includes a first curved portion that faces one of the side edges, and the second hook-shaped member includes a second curved portion that faces the other of the side edges.

5. A device according to claim 1, and further comprising a second opening formed in the base member for receiving the catheter.

6. A device according to claim 5, wherein the second opening comprises a circular edge and a pair of converging edges that extend from a front edge of the base member to the circular edge.

7. A device according to claim 6, and further comprising at least one alignment mark extending toward a radial center of the circular edge so that the base member can be aligned with a body insertion point of the catheter.

8. A device according to claim 6, and further comprising an adhesive strip positioned on the base member for covering the second opening and securing the catheter to the base member.

9. A device according to claim 5, and further comprising at least one generally laterally extending alignment mark adjacent the second opening so that the base member can be aligned with a body insertion point of the catheter.

10. A device according to claim 5, and further comprising an adhesive strip positioned on the base member for covering the second opening and securing the catheter to the base member.

11. A device according to claim 1, wherein the base member is constructed of sheet material.

12. A device according to claim 9, wherein the base member and the hooks are integrally formed of the sheet material.

13. A device according to claim 11, wherein the sheet material is flexible to accommodate a shape of the body portion.

14. A device according to claim 11, wherein the sheet material is sufficiently transparent to view the body portion under the base member when installed on the body portion.

15. A device according to claim 1, and further comprising a second strap connected to the base member, the second strap being adapted for wrapping around the body member to thereby secure the base member and the catheter to the body member in conjunction with the first strap.

16. A device according to claim 15, wherein the first strap is positioned on the base member forward of the first opening, and the second strap is positioned on the base member rearward of the first opening.

17. A device for securing an intravenous (IV) catheter inserted into a body portion of a patient, the IV catheter being attached to an IV line, the device comprising:
a generally longitudinal, substantially planar base member adapted for placement on a body portion of a patient;
a first opening formed in the base member, the first opening including a front edge, a rear edge, and a pair of side edges extending between the front and rear edges;
a first hook-shaped member extending generally longitudinally into the opening from the front edge;
a second hook-shaped member extending generally longitudinally into the opening from the rear edge in opposing relationship to the first hook-shaped member, the hook-shaped members being adapted to receive and hold the IV line against movement;
a second opening formed in the base member for receiving the catheter, the second opening comprising a circular edge and a pair of converging edges that extend from a front edge of the base member to the circular edge;
first and second straps connected to the base member, the first and second straps being adapted for wrapping around the body member to thereby secure the base member and the catheter to the body member; and
an adhesive strip positioned on the base member for covering the second opening and securing the catheter to the base member.

18. A device according to claim 17, wherein the first strap is positioned on the base member forward of the first opening, and the second strap is positioned on the base member rearward of the first opening.

19. A device according to claim 17, wherein the base member is constructed of a sheet material.

20. A device according to claim 19, wherein the base member and the hooks are integrally formed of the sheet material.

21. A device according to claim 19, wherein the sheet material is flexible to accommodate a shape of the body portion.

22. A device according to claim 19, wherein the sheet material is sufficiently transparent to view the body portion under the base member when installed on the body portion.

23. A device according to claim 17, wherein the first hook-shaped member includes a first curved portion that faces one of the side edges, and the second hook-shaped member includes a second curved portion that faces the other of the side edges.
